SEQUENCE LISTING (6 sequences)

SEQ ID NO: 1 - DNA, 20 bp, Artificial Sequence
  ICP0 Forward Primer
atgtttcccgtctggtccac

SEQ ID NO: 2 - DNA, 19 bp, Artificial Sequence
  ICP0 Reverse Primer
ccctgtcgccttacgtgaa

SEQ ID NO: 3 - DNA, 23 bp, Artificial Sequence
  ICP0 Probe
ccccgtctccatgtccaggatgg

SEQ ID NO: 4 - DNA, 20 bp, Artificial Sequence
  18S RNA Forward Primer
cggctaccacatccaaggaa

SEQ ID NO: 5 - DNA, 18 bp, Artificial Sequence
  18S RNA Reverse Primer
gctggaattaccgcggct

SEQ ID NO: 6 - DNA, 22 bp, Artificial Sequence
  18S RNA Probe
tgctggcaccagacttgccctc

(19) United States
(12) Patent Application Publication
(10) Pub. No.: US 2011/0171257 A1
(43) Pub. Date: Jul. 14, 2011

(54) HERPES SIMPLEX VIRUS AMPLICON VECTORS DERIVED FROM PRIMARY ISOLATES

(75) Inventors: Stephen Dewhurst, Rochester, NY (US); William J. Bowers, Webster, NY (US); Howard J. Federoff, Bethesda, MD (US); John G. Frelinger, Pittsford, NY (US); Michael C. Keefer, Rochester, NY (US)

(73) Assignee: UNIVERSITY OF ROCHESTER, Rochester, NY (US)

(21) Appl. No.: 12/602,818

(22) PCT Filed: Jun. 3, 2008
(86) PCT No.: PCT/US08/65640

§ 371 Date: Jun. 3, 2010

Related U.S. Application Data
(60) Provisional application No. 60/941,849, filed on Jun. 4, 2007.

(51) Int. Cl. A61K 39/21; C12N 15/63; C12P 19/34; A61K 39/00; A61P 35/00; A61P 31/18; A61P 25/28; C12Q 1/70

(52) U.S. Cl. 424/208.1; 435/320.1; 435/91.1; 424/277.1; 424/184.1; 435/5

(57) ABSTRACT

Provided herein are HSV amplicon particles and methods of making and using HSV amplicon particles. The particles are generated using primary HSV isolates or packaging vectors derived from primary HSV isolates.

### CROSS-REFERENCE TO RELATED APPLICATION

This application claims priority to U.S. Provisional Application No. 60/941,849, filed Jun. 4, 2007, which is incorporated herein by reference in its entirety.

### STATEMENT REGARDING FEDERALLY FUNDED RESEARCH

This invention was made with government support under Grant Nos. F31 AI054330, T32 CA009363 and P01 AI056356 awarded by the National Institutes of Health. The government has certain rights in this invention.

### BACKGROUND

HSV-1 amplicon vectors are useful for multiple gene transfer applications including vaccine delivery. However, current methods for the generation of amplicon stocks rely on the use of highly passaged helper virus strains in order to produce infectious amplicon particles (Geller and Breakefield, Science 241:1667-9, 1988; Logvinoff and Epstein, Hum Gene Ther 12:161-7, 2001), or (in the case of helper-free amplicon stocks) on the use of molecularly cloned helper virus genomes that have been derived from laboratory-adapted strains (Fraefel et al., J Virol 70:7190-7, 1996; Saeki et al., Mol Ther 3:591-601, 2001; Saeki et al., Hum Gene Ther 9:2787-94, 1998; Stavropoulos and Strathdee, J Virol 72:7137-43, 1998).

Serial passage of herpesviruses in cultured cell lines is known to result in profound changes in the virus genome, including point mutations, alterations of splicing patterns and even deletions of large segments of viral DNA. This is exemplified by the genetic changes and associated loss of virulence that characterize the serially passaged Oka vaccine strain of varicella-zoster virus, when compared to the parental Oka strain. Similarly, laboratory-adapted strains of human cytomegalovirus (HCMV), such as the AD169 strain, possess an extensive genetic deletion (encompassing approximately 15 kb of the viral DNA genome) when compared to primary isolates. Since HCMV strains are typically propagated in fibroblasts, this presumably explains why laboratory adapted strains have lost the ability to infect endothelial cells, when compared to primary isolates.

Genes which are most prone to mutation following prolonged passage of HCMV in cell culture often have roles in pathogenicity or tropism. Therefore, changes in the genetic composition and biological properties of herpesviruses following adaptation to laboratory culture conditions are likely to result in loss of properties that may be desirable in the context of vaccine delivery and/or amplicon generation. For example, laboratory-adapted strains of HCMV not only lose the ability to infect endothelial cells, but also lose the ability to efficiently infect primary dendritic cells.

### SUMMARY

Provided herein are HSV amplicon particles and methods of making and using HSV amplicon particles. The particles are generated using primary HSV isolates or packaging vectors derived from primary HSV isolates. For example, provided herein is a helper-free amplicon particle that include an amplicon vector and packaging components derived from a primary HSV isolate. Further provided are methods of making the particle by co-transfecting a host cell with an amplicon vector comprising an HSV origin of replication and an HSV cleavage/packaging signal and at least one packaging vector, wherein the packaging vector is a derivative of a primary HSV isolate, wherein the co-transfection step is performed under conditions that result in production of the HSV amplicon particles in the host cell. Also provided is a method of selecting a primary isolate for use in the methods of making the amplicon particles. Methods of using the particles include methods of treating cancer, methods of treating a disease caused by an infectious agent and methods of treating an aggregated disorder by administering to a subject an amplicon particle disclosed herein. Also provided are cells containing one or more of the amplicon particles and kits for using the amplicon particles.

The details of one or more aspects are set forth in the accompanying drawings and the description below. Other features, objects, and advantages will be apparent from the description and drawings, and from the claims.

### DESCRIPTION OF DRAWINGS

FIGS. 1A and 1B are graphs showing that amplicon stocks propagated by most primary HSV-1 isolates are more efficient at transducing continuous cells than stocks generated using the packaging strain F5, which was derived from the molecularly cloned genome of a lab-adopted isolate, HSV-1 strain 17. FIG. 1A shows VERO cells and FIG. 1B shows HEK 293 A cells transduced at a multiplicity of infection (MOI) of 0.1 with HSV-1 amplicon particles packaged in the presence of the various isolates. Transduced cells were assayed for total β-galactosidase activity 24 hours post infection. Cleared lysates (1 μg of total protein) were assayed for activity. Data represent mean values calculated from three replicate measurements. Measurements of β-galactosidase activity were normalized to total protein. Bars denote the standard deviation of the three individual values. The data show that amplicon stocks packaged by a majority of the primary isolates were more efficient at transducing both VERO and 293 cells than stocks that were generated using the packaging strain F5.

FIGS. 2A, 2B and 2C are graphs showing that amplicon stocks propagated by most primary HSV-1 isolates are more efficient at transducing dendritic cells than stocks generated using the packaging strain F5. Human monocyte-derived dendritic cells (DC) from three different donors were transduced at an MOI of 0.1 with HSV-1 amplicon particles packaged in the presence of the various isolates. FIGS. 2A, 2B and 2C show amplicon-transduced human DC assayed for total β-galactosidase activity 24 hours post transduction. Cleared lysates (1 μg total protein) were assayed for β-galactosidase activity. Data represent mean values calculated from three replicate measurements. Measurements of β-galactosidase activity were normalized to total protein. Bars denote the standard deviation of the three individual values.

FIG. 3 is a micrograph showing the analysis of amplicon-mediated transduction of dendritic cells using X-gal histochemistry. Human monocyte-derived DC were transduced at an MOI of 0.1 with lacZ-encoding HSV-1 amplicon particles packaged in the presence of the various isolates, and then assayed by X-gal histochemistry 24 hours post transduction. Staining was performed as described in the Examples below and stained cells were observed by phase contrast light microscopy. Representative results for one donor are shown. The data show that amplicon stocks packaged by the primary isolates were more efficient at transducing primary human dendritic cells than stocks that were generated using the packaging strain F5. Numbers indicate the percentage of lacZ-positive cells within each culture.

FIGS. 4A, 4B and 4C are scatterplots showing linear regression analyses for cell transduction data. Linear regression analysis of cell transduction data, for the VERO and 293 cell lines, and the primary dendritic cells. The associations between gene expression levels were examined in a pairwise fashion for the three different cell types using linear regression and correlation analysis. The figure shows the scatterplots and the computed least-squares regression line (GraphPad Prism). The data that were used in these analyses correspond to the datasets shown in FIGS. 2A, 2B, 2C and FIG. 3 (DC Batch 1). There was a very strong correlation between the magnitude of amplicon-mediated transduction in the two cultured cell lines (293, VERO), but a weaker correlation between transduction efficiency in these cell lines and amplicon-mediated gene expression in primary dendritic cells. A summary of the statistical analyses corresponding to these plots is provided in Table 2.

FIG. 5 is a graph showing that representative primary HSV-1 strains and lab-adapted virus stocks replicate with approximately equivalent kinetics in VERO cells. VERO cells were infected with 4×10⁴ pfu (MOI=0.2) of two representative HSV-1 clinical isolates (10, 19), as well as the molecularly cloned F5 viral stock, and two laboratory-adapted strains (17 and KOS). The cultures were then sampled at selected time points (0, 2, 6, 12, 18, 24, 36, 48 hours), and viral genomic titers in cell lysates were measured by quantitative real-time PCR amplification, using oligonucleotide primers specific for the ICP0 gene. Viral genome titers were normalized in terms of the virus DNA load per 12.5 ng of total input cellular DNA. The results show that the virus strains replicated with essentially indistinguishable kinetics, with the exception of the F5 strain.

FIG. 6 is a graph showing that amplicon stocks packaged by representative primary HSV-1 strains and lab-adapted virus stocks differ in their ability to transduce 293 cells. HEK 293 A cells were transduced at an MOI of 0.1 with HSV-1 amplicon particles packaged by the various isolates, and then cultured in the presence or absence of acyclovir (1 μM). Transduced cells were assayed for total β-galactosidase activity 24 hours post infection. Cleared lysates (1 μg of total protein) were assayed for activity. Data represent mean values calculated from three replicate measurements. Measurements of β-galactosidase activity were normalized to total protein. Bars denote the standard deviation of the three individual values. The data show that acyclovir had no significant effect on amplicon-mediated gene expression, when measured at this early time point. The data also show that amplicons packaged by lab-adapted virus strains (KOS and 17+) and primary isolate 19 were efficient at transducing 293 cells, whereas amplicons packaged by the molecularly cloned F5 virus and primary isolate 10 were inefficient at transducing this cell line.

FIGS. 7A and 7B are graphs showing that amplicon stocks packaged by representative primary HSV-1 strains and lab-adapted virus stocks differ in their ability to transduce primary dendritic cells (DC). DC were transduced at an MOI of 0.1 with HSV-1 amplicon particles packaged by the various isolates. Transduced cells were assayed for total β-galactosidase activity 24 hours post infection. Cleared lysates (1 μg of total protein) were assayed for activity. Data represent mean values calculated from three replicate measurements. Measurements of β-galactosidase activity were normalized to total protein. Bars denote the standard deviation of the three individual values. The data show results for DC prepared from two different donors (FIG. 7A and FIG. 7B). Amplicons packaged by the parental, lab-adapted strain 17 and its molecularly cloned counterpart F5 were both poor at transducing DC. In contrast, amplicons packaged by the KOS strain and primary isolate 19 were much more efficient at transducing DC. Note that, for both donors, results for primary isolate 19 are significantly different (better) than those for strain KOS (p<0.01 in both cases; one-way ANOVA with Tukey's post-test).

FIG. 8A shows the structure of a wild-type HSV-1 virus at the UL41 gene locus (top) and of a BAC 8 construct (bottom). The recombination plasmid used to generate this BAC was homologous to UL41 and replaced this nonessential gene with the GFP reporter gene giving rise to diagnostic BamHI restriction fragments that are shown schematically. FIG. 8B is a picture of a gel showing restriction digest of two BAC 8 clones. FIG. 8C is a micrograph showing that both clones were infectious following transfection into VERO cells.

FIG. 9A is a graph showing the percent of mCD40L positive chronic lymphocytic leukemia cells after transduction with HSV amplicon vectors encoding mCD40L packaged using different HSV-1 helper bacmids. FIG. 9B is a graph showing mean fluorescence intensity of CD40L staining on chronic lymphocytic leukemia cells after transduction with HSV amplicon vectors encoding mCD40L packaged using different HSV-1 helper bacmids.

FIG. 10A is a graph showing the percent of CD86 positive chronic lymphocytic leukemia cells after transduction with HSV amplicon vectors encoding CD86 packaged using different HSV-1 helper bacmids. FIG. 10B is a graph showing mean fluorescence intensity of CD86 staining on chronic lymphocytic leukemia cells after transduction with HSV amplicon vectors encoding CD86 packaged using different HSV-1 helper bacmids.

### DETAILED DESCRIPTION

Herpes Simplex Virus Type-1 (HSV-1) amplicon vectors are being explored for a wide range of potential applications, including vaccine delivery and immunotherapy of cancer. While extensive effort has been directed toward the improvement of the amplicon payload in these vectors, little attention has been paid to the effect of the packaging HSV-1 strains on the biological properties of co-packaged amplicon vectors. Current methods for the generation of helper-free HSV-1 amplicon stocks involve the transient transfection of amplicon plasmid DNA into packaging-permissive cells [i.e., baby hamster kidney cells (BHK) or 2-2 cells], together with a bacmid construct that contains a non-packageable HSV-1 genome. The biological properties of molecularly cloned virus genomes remain incompletely characterized and may not be ideal for vaccine applications and/or efficient production of amplicon stocks. To this end, experiments were conducted to compare the properties of a reconstituted infectious virus stock derived from the original HSV-1 cosmid panel (designated herein as F5), with those of a set of 19 clinical HSV-1 isolates that had been only minimally passaged, and two additional laboratory-adapted HSV-1 isolates (KOS and strain 17+, which is the parental virus from which the molecularly cloned F5 stock was derived). As described in the Examples below, there was variability in the efficiency with which amplicon stocks packaged by these viruses were able to transduce established cell lines and primary human dendritic cells (DC). However, amplicon stocks generated using the minimally passaged primary isolates outperformed the F5-based stock. Moreover, amplicons packaged by both the molecularly cloned F5 virus and its lab-adapted parent (strain 17) were equally inefficient at transducing DC, suggesting that this property is intrinsic to strain 17 and not an artifact of the molecular cloning process. These data show that minimally passaged, primary HSV-1 isolates can be used for the production of amplicon vector stocks for use as vaccines and gene therapy.

Provided herein are amplicon-based systems and methods for making amplicon-based systems. These systems include helper free and helper containing systems. Amplicon vectors are dependent upon helper virus function to provide the replication machinery and structural proteins necessary for packaging amplicon plasmid DNA into HSV amplicon particles. Helper-containing systems include amplicon vectors or plasmids packaged, for example, by a replication-defective virus that lacks an essential viral regulatory gene. The final product of helper-containing virus-based packaging system contains a mixture of varying proportions of helper and amplicon particles. Helper-free amplicon packaging systems were developed by providing a packaging-deficient helper virus genome via one or more cosmids or by using one or more bacterial artificial chromosomes (BAC) that encode for the entire HSV genome minus its cognate cleavage/packaging signals.

Helper virus-free systems for making HSV amplicon particles, including those described herein, include the use of at least one vector, referred to herein as a packaging vector, that, upon delivery to a cell that supports HSV replication, expresses sufficient structural HSV proteins that are capable of assembling amplicon vectors into HSV amplicon particles. Sets of cosmids have been isolated that contain overlapping clones that represent the entire genomes of a variety of herpesviruses (see U.S. Pat. No. 5,998,208). The packaging vectors are prepared so that none of the viruses used will contain a functional HSV cleavage-packaging site containing sequence. This sequence is referred to as the “a” sequence (and is not encoded by the packaging vector(s)). The “a” sequence can be deleted from the packaging vector(s) by any of a variety of techniques practiced by those of ordinary skill in the art. For example, the entire sequence can be deleted by, for example, the techniques described in U.S. Pat. No. 5,998,208. Alternatively, a sufficient portion of the “a” sequence can be deleted to render it incapable of packaging. Another alternative is to insert nucleotides into the site that render the “a” sequence non-functional.

An HSV amplicon particle consists of four components, the envelope, the tegument, the capsid and the particle genome. The core of the HSV amplicon particle that contains the particle genome or amplicon vector is formed from a variety of structural genes that create the capsid. The genes for capsid formation must be present in a host cell used to prepare HSV amplicon particles, whether the genes are expressed from the host cell genome or on a packaging vector. Optionally, the necessary envelope proteins are also expressed from the host cell genome or the packaging vector. In addition, there are a number of other proteins present on the surface of a herpesvirus particle. Some of these proteins help mediate viral entry into certain cells. Thus, the inclusion or exclusion of the functional genes encoding these proteins depend upon the particular use of the particle. As used herein, the phrase packaging components, refers to the envelope, the tegument and the capsid of the HSV amplicon particle.

Provided herein are HSV amplicon particles and a method for producing HSV amplicon particles. Also provided are HSV amplicon particles made by the provided methods and cells comprising the HSV amplicon particles. The particles are generated using primary HSV isolates or packaging vectors derived from primary HSV isolates. The one or more packaging vectors individually or collectively encode all essential HSV genes but exclude all cleavage/packaging signals. As used herein, the phrase packaging vectors derived from primary HSV isolates means that the essential HSV genes are obtained or derived from the primary HSV isolate. Thus, the packaging vectors individually or collectively encode the essential HSV genes obtained or derived from a primary HSV isolate. Such HSV amplicon particles have increased cell tropism and/or infectivity as compared to control HSV amplicon particles. Thus, the provided HSV amplicon particles can have at least 5-fold, 10-fold, 20-fold, or more, or any amount between 5-fold and 20-fold increased infectivity as compared to a control HSV amplicon particle. In addition, the provided HSV amplicon particles have increased cell tropism, (i.e., expanded host range) as compared to a control HSV amplicon particle. As used herein, increased cell tropism means that the provided HSV amplicon particles can infect cells that are minimally infected or not infected by control HSV amplicon particles. Thus, the provided HSV amplicon particles can infect a larger number of biologically relevant cell types such as, for example, dendritic cells, neurons, tumor cells and the like.

Optionally, the primary HSV isolates are minimally passaged. As used herein, the term passaged refers to the serial propagation of HSV in cultured cell lines. Serial propagation of the HSV isolates can result in phenotypic and molecular adaptation of the virus to these cultured cell lines. As used herein, the phrase primary HSV isolate refers to an HSV isolate that is not a laboratory-adapted strain of HSV or has not been serially propagated in cultured cell lines. As used herein, a primary HSV isolate that has not been serially propagated refers to an HSV isolate that has been passaged about 10 times or less in cultured cell lines. Thus, the primary HSV isolate has been serially passaged between 0 to about 10 times or any number of times between 0 and 10. Thus, for example a primary HSV isolate can be passaged 0 times, 1 time, 3 times, 5 times, 7 times or up to about 10 times or any number of times in between 0 and 10. The primary HSV isolate is selected based on its ability to produce HSV amplicon particles that transduce, for example, dendritic cells. As used herein, control HSV amplicon particles refers to HSV amplicon particles that are not made using primary HSV isolates or packaging vectors derived from primary HSV isolates. Thus, for example, a control HSV amplicon particle can be made using a laboratory-adapted HSV isolate.

The provided HSV amplicon particle comprises an amplicon vector and packaging components, wherein the packaging components are derived from a primary HSV isolate. Optionally, the HSV amplicon particle is helper-free. The primary HSV isolate is selected based on its ability to produce amplicon particles that transduce dendritic cells. The packaging components usually include an envelope, a tegument and a capsid. As described in more detail below, the amplicon vector can also comprise an expressible transgene.

The method for producing HSV amplicon particles comprises co-transfecting a host cell with an amplicon vector and a primary HSV isolate or one or more packaging vectors derived from a primary HSV isolate. The co-transfection step is performed under conditions that result in production of HSV amplicon particles in the host cell. Optionally, the HSV amplicon particles can be isolated from the host cell. The amplicon vector can comprise an HSV origin of replication and an HSV cleavage/packaging signal. Optionally, the amplicon vector comprises an expressible heterologous transgene. The one or more packaging vectors individually or collectively encode all essential HSV genes but exclude all cleavage/packaging signals. Thus, the packaging vectors individually or collectively encode the essential HSV genes obtained or derived from a primary HSV isolate. The packaging vector optionally lack an ori_{L} origin of replication. The packaging vectors can comprise a vhs expression vector encoding a virion host shutoff protein. When the amplicon vector includes a transgene, the HSV amplicon particles thus include the transgene.

The amplicon vector can be any HSV amplicon vector which includes an HSV origin of replication, an HSV cleavage/packaging signal, and, optionally, a heterologous transgene expressible in a subject. The amplicon vector can also include a selectable marker gene and/or an antibiotic resistance gene.

The HSV cleavage/packaging signal can be any suitable cleavage/packaging signal such that the vector can be packaged into a HSV amplicon particle that is capable of adsorbing to a cell (i.e., which is to be transformed or transduced). A suitable cleavage/packaging signal is the HSV-1 “a” segment located at approximately nucleotides 127-1132 of the a sequence of the HSV-1 virus or its equivalent (Davison et al., “Nucleotide sequences of the joint between the L and S segments of herpes simplex virus types 1 and 2,” J. Gen. Virol. 55:315-331 (1981), which is hereby incorporated by reference in its entirety, at least for its disclosure relating to cleavage/packaging signals). There are a variety of sequences related to, for example, HSV-1 “a” and other HSV genes that are disclosed on GenBank, at www.pubmed.gov, and these sequences and others are herein incorporated by reference in their entireties as well as for individual subsequences contained therein. For example, the HSV-1 “a” sequence can be found at GenBank Accession Nos. K03357, M10963, M13884 and M13885.

The HSV origin of replication can be any suitable origin of replication that allows for replication of the amplicon vector in the host cell used for replication and packaging of the vector into the HSV amplicon particles. A suitable origin of replication is the HSV-1 “c” region which contains the HSV-1 oriₛ segment located at approximately nucleotides 47-1066 of the HSV-1 virus or its equivalent (McGeogh et al., Nucl. Acids Res. 14:1727-1745 (1986), which is hereby incorporated by reference at least for its disclosure relating to HSV origins of replication). Origin of replication signals from other related viruses (e.g., HSV-2) can also be used.

Selectable marker genes are known in the art and include, without limitation, galactokinase, beta-galactosidase, chloramphenicol acetyltransferase, beta-lactamase, green fluorescent protein (GFP), and alkaline phosphate. Antibiotic resistance genes are known in the art and include, without limitation, ampicillin, streptomycin, and spectromycin.

Amplicon vectors include, but are not limited to, pHSVlac (ATCC Accession 40544; U.S. Pat. No. 5,501,979 to Geller et al.; Stavropoulos and Strathdee, An enhanced packaging system for helper-dependent herpes simplex virus vectors, J. Virol., 72:7137-43 (1998)) and pHENK (U.S. Pat. No. 6,040,172 to Kaplitt et al.). The pHSVlac vector includes the HSV-1 “a” segment, the HSV-1 “c” region, an ampicillin resistance marker, and an E. coli lacZ marker. The pHENK vector includes the HSV-1 “a” segment, an HSV-1 on segment, an ampicillin resistance marker, and an E. coli lacZ marker under control of the promoter region isolated from the rat preproenkephalin gene (i.e., a promoter operable in brain cells).

Amplicon vectors can be modified by introducing therein, at an appropriate restriction site, either a complete transgene which has already been assembled, or a coding sequence can be ligated into an empty amplicon vector that already contains appropriate regulatory sequences (promoter, enhancer, polyadenylation signal, transcription terminator, etc.) positioned on either side of the restriction site where the coding sequence is to be inserted, thereby forming the transgene upon ligation. Alternatively, when using the pHSVlac vector, the lacZ coding sequence can be excised using appropriate restriction enzymes and replaced with a coding sequence for the transgene.

Suitable transgenes will include one or more appropriate promoter elements capable of directing the initiation of transcription by RNA polymerase, optionally one or more enhancer elements, and suitable transcription terminators or polyadenylation signals. The promoter elements are selected such that the promoter will be operable in the cells which are ultimately intended to be transformed. A number of promoters have been identified which are capable of regulating expression within a broad range of cell types. These include, without limitation, HSV immediate-early 4/5 (1E4/5) promoter, cytomegalovirus (CMV) promoter, SV40 promoter, β-actin promoter, other ubiquitous viral and cellular promoters and synthetic promoter/enhancer elements. Synthetic promoter/enhancer elements can be comprised of concatenated transcription factor binding sites and associated initiation elements. Likewise, a number of other promoters have been identified which are capable of regulating expression within a narrow range of cell types. These include, without limitation, neural-specific enolase (NSE) promoter, tyrosine hydroxylase (TH) promoter, GFAP promoter, preproenkephalin (PPE) promoter, myosin heavy chain (MHC) promoter, insulin promoter, cholineacetyltransferase (CHAT) promoter, dopamine β-hydroxylase (DBH) promoter, calmodulin dependent kinase (CamK) promoter, c-fos promoter, c-jun promoter, vascular endothelial growth factor (VEGF) promoter, erythropoietin (EPO) promoter, and EGR-1 promoter. Suitable promoters also include promoters active in cells of hematopoietic lineage including dendritic cells and macrophages such as, for example, CD11b, CD11c, CD83, Fascin and MHC class II promoters.

The transcription termination signal, likewise, is selected such that it is operable in the cells which are ultimately intended to be transformed. Suitable transcription termination signals include, without limitation, polyA signals of HSV genes such as the vhs polyadenylation signal, SV40 polyA signal, and CMV IE1 polyA signal.

The HSV amplicon particles described herein (and the cells that contain them) can express a heterologous protein (i.e., a full-length protein or a portion thereof (e.g., a functional domain or antigenic peptide) that is not naturally encoded by a herpesvirus). The heterologous protein can be any protein that conveys a therapeutic benefit to the cells in which it, by way of infection with an HSV amplicon particle, is expressed or to a subject who is treated with those cells. Thus, the amplicon vector can comprise an expressible transgene. Preferably, the transgene encodes a therapeutic product. Suitable therapeutic products include, but are not limited to, proteins, antigens or RNA molecules. Suitable RNA molecules, include, but are not limited to, antisense RNA, RNAi, and an RNA ribozyme. Suitable antigens include, but are not limited to, tumor-specific antigens, antigens of an infectious agent and antigens of a protein aggregate. Tumor-specific antigens include prostate cancer tumor-specific antigens, breast cancer-specific antigens, melanoma antigens and other antigens expressed by tumor cells or cancerous tissues.

In addition, the therapeutic products can be immunomodulatory (e.g., immunostimulatory) proteins (as described in U.S. Pat. No. 6,051,428). For example, the heterologous protein can be an interleukin (e.g., IL-1, IL-2, IL-4, IL-10, or IL-15), an interferon (e.g., IFNγ), a granulocyte macrophage colony stimulating factor (GM-CSF), a tumor necrosis factor (e.g., TNFα), a chemokine (e.g., RANTES, MCP-1, MCP-2, MCP-3, DC-CK1, MIP-1α, MIP-3α, MIP-β, MTP-3β, an α or C-X-C chemokine (e.g., IL-8, SDF-1β, 8DF-1α, GRO, PF-4 and MIP-2). Other chemokines that can be usefully expressed are in the C family of chemokines (e.g., lymphotactin and CX3C family chemokines).

Intercellular adhesion molecules are transmembrane proteins within the immunoglobulin superfamily that act as mediators of adhesion of leukocytes to vascular endothelium and to one another. The vectors described herein can be made to express ICAM-1 (also known as CD54) and/or another cell adhesion molecule that binds to T or B cells (e.g., ICAM-2 and ICAM-3).

Costimulatory factors that can be expressed by the vectors described herein are cell surface molecules, other than an antigen receptor and its ligand, that are required for an efficient lymphocytic response to an antigen (e.g., B7 (also known as CD80) and CD40L).

Therapeutic RNA molecules include, without limitation, antisense RNA, inhibitory RNA (RNAi), and an RNA ribozyme. The RNA ribozyme can be either cis or trans acting, either modifying the RNA transcript of the transgene to afford a functional RNA molecule or modifying another nucleic acid molecule. Exemplary RNA molecules include, without limitation, antisense RNA, ribozymes, or RNAi to nucleic acids for huntingtin, alpha synuclein, scatter factor, amyloid precursor protein, p53, and VEGF.

Therapeutic proteins include, without limitation, receptors, signaling molecules, transcription factors, growth factors, apoptosis inhibitors, apoptosis promoters, DNA replication factors, enzymes, structural proteins, neural proteins, and histone or non-histone proteins. Exemplary protein receptors include, without limitation, all steroid/thyroid family members, nerve growth factor (NGF), brain derived neurotrophic factor (BDNF), neutotrophins 3 and 4/5, glial derived neurotrophic factor (GDNF), cilary neurotrophic factor (CNTF), persephin, artemin, neurturin, bone morphogenetic factors, c-ret, gp 130, dopamine receptors (D 1D5), muscarinic and nicotinic cholinergic receptors, epidermal growth factor (EGF), insulin and insulin-like growth factors, leptin, resistin, and orexin. Exemplary protein signaling molecules include, without limitation, all of the above-listed receptors plus MAPKs, ras, rac, ERKs, NFK1β, GSK3β, AKT, and PI3K. Exemplary protein transcription factors include, without limitation, CBP, HIF-1α, NPAS1 and 2, H1F-1β, p53, p73, nun 1, nurr 77, MASHs, REST, and NCORs. Exemplary neural proteins include, without limitation, neurofilaments, GAP-43, SCG-10, etc. Exemplary enzymes include, without limitation, TH, DBH, aromatic amino acid decarboxylase, parkin, unbiquitin E3 ligases, ubiquitin conjugating enzymes, cholineacetyltransferase, neuropeptide processing enzymes, dopamine, VMAT and other catecholamine transporters. Exemplary histones include, without limitation, H1-5. Exemplary non-histones include, without limitation, ND10 proteins, PML, and HMG proteins. Exemplary pro-and anti-apoptotic proteins include, without limitation, bax, bid, bak, bcl-xs, bcl-xl, bcl-2, caspases, SMACs, and IAPs.

The one or more packaging vectors used in the provided methods individually or collectively encoding all essential HSV genes from a primary HSV isolate but excluding all cleavage/packaging signals can either be in the form of a set of vectors or a single bacterial-artificial chromosome (BAC), which is formed, for example, by combining the set of vectors to create a single, doublestranded vector. The BAC can include a pac cassette inserted at a BamHI site located within the UL41 coding sequence, thereby disrupting expression of the HSV-1 virion host shutoff protein.

As used herein, the phrase essential HSV genes, includes all genes that encode polypeptides that are necessary for replication of the amplicon vector and structural assembly of the HSV amplicon particles. Thus, in the absence of such genes, the HSV amplicon vector is not properly replicated and packaged within a capsid to form an amplicon particle capable of adsorption. Such essential HSV genes have previously been reported in review articles by Roizman, Proc. Natl. Acad. Sci. USA 11:307-113, 1996 and Roizman, Acta Virologica 43:75-80, 1999. Another source for identifying such essential genes is available at the Internet site operated by the Los Alamos National Laboratory, Bioscience Division, which reports the entire HSV-1 genome and includes a table identifying the essential HSV-1 genes. These references are incorporated herein in their entireties at least for essential genes.

As described above, the packaging vectors are derivatives of a primary HSV isolate. Thus, the genes that encode polypeptides necessary for replication of the amplicon vector and structural assembly of the HSV amplicon particles are derived from a primary HSV isolate. Preferably, the primary HSV isolate is selected based on its ability to produce HSV amplicon particles that transduce dendritic cells. Thus, provided is a method for selecting a primary HSV isolate for use in the methods described herein comprising co-transfecting a host cell with an amplicon vector comprising an HSV origin of replication and an HSV cleavage/packaging signal and a candidate primary HSV isolate to be tested, under conditions that allow production of at least one HSV amplicon particle in the host cell, isolating the amplicon particle from the host cell, contacting the amplicon particle with at least one dendritic cell, and determining whether the amplicon particle transduces the dendritic cell. Transduction of the dendritic cell by the amplicon particle indicates that the primary HSV isolate is suitable for use in the methods described herein.

The provided HSV amplicon particles isolated from host cells are included in a composition with a suitable carrier. The HSV amplicon particles may also be administered in injectable dosages by dissolution or suspension of these materials in a physiologically acceptable diluent with a pharmaceutical carrier. Such carriers include sterile liquids, such as water and oils, with or without the addition of a surfactant and other pharmaceutically and physiologically acceptable carriers, including adjuvants, excipients or stabilizers. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

For use as aerosols, HSV amplicon particles, in solution or suspension, may be packaged in a pressurized aerosol container together with suitable propellants, for example, hydrocarbon propellants like propane, butane, or isobutane with conventional adjuvants. The HSV amplicon particles also may be administered in a non-pressurized form such as in a nebulizer or atomizer.

The exact amount of the compositions required will vary from subject to subject, depending on the species, age, weight and general condition of the subject, the severity of the disease being treated, the particular virus or vector used, its mode of administration and the like. Thus, it is not possible to specify an exact amount for every composition. However, an appropriate amount can be determined by one of ordinary skill in the art using only routine experimentation given the teachings herein. Typically, a composition will contain at least about 1×10⁷ amplicon particles/ml, together with the carrier, excipient, and/or stabilizer. Titers can be higher, however. For example, titers can be 1×10⁸ to 5×10⁸, or even higher (e.g., 1×10⁹ to 5×10⁹). The titer can be any amount in between 1×10⁷ to 1×10¹⁰.

Also provided are kits comprising the HSV amplicon particles described herein and kits for preparing HSV amplicon particles. A kit for preparing HSV amplicon particles comprises an amplicon vector comprising an HSV origin of replication and an HSV cleavage/packaging signal and at least one packaging vector, wherein the packaging vector is a derivative of a primary HSV isolate and/or a primary HSV isolate for producing a packaging vector. A kit can further include instructions for use, a container, an administrative means (e.g., a syringe), other biologic components such as one or more cells and the like. The amplicon vectors and packaging vectors can comprise one or more of the components described herein.

Provided are methods of treating diseases in a subject comprising administering to the subject the HSV amplicon particles described herein. Preferably, the HSV amplicon particles comprise an expressible transgene. Diseases to be treated by the provided methods include, but are not limited to cancer, diseases caused by infectious agents and protein aggregate disorders.

The compositions disclosed herein (including HSV amplicon particles and cells that contain them) can be used to treat patients who have been, or who may become, infected with a wide variety of agents (including viruses such as a human immunodeficiency virus, human papilloma virus, herpes simplex virus, influenza virus, pox viruses, bacteria, such as E. coli or a Staphylococcus, or a parasite) and with a wide variety of cancers such as, for example, prostate cancer. A subject can be treated after they have been diagnosed as having a cancer or an infectious disease or, since the agents can be formulated as vaccines, subjects can be treated before they have developed cancer or contracted an infectious disease. Thus, the term treatment encompasses prophylactic treatment. Prophylactic treatments include delaying or reducing one or more symptoms or clinical signs of the disease or disorder to be treated.

Neuronal diseases or disorders and protein aggregate disorders that can be treated include lysosomal storage diseases (e.g., by expressing MPS1-VIII, hexoaminidase A/B, etc.), Lesch-Nyhan syndrome (e.g., by expressing HPRT), amyloid polyneuropathy (e.g., by expressing β-amyloid converting enzyme (BACE) or amyloid antisense), Alzheimer's Disease (e.g., by expressing NGF, CHAT, BACE, etc.), retinoblastoma (e.g., by expressing pRB), Duchenne's muscular dystrophy (e.g., by expressing Dystrophin), Parkinson's Disease (e.g., by expressing GDNF, Bcl-2, TH, AADC, VMAT, antisense to mutant α-synuclein, etc.), Diffuse Lewy Body disease (e.g., by expressing heat shock proteins, parkin, or antisense or RNAi to α-synuclein), stroke (e.g., by expressing Bcl-2, HIF-DN, BMP7, GDNF, other growth factors), brain tumor (e.g., by expressing angiostatin, antisense VEGF, antisense or ribozyme to EGF or scatter factor, pro-apoptotic proteins), epilepsy (e.g., by expressing GAD65, GAD67, pro-apoptotic proteins into focus), or arteriovascular malformation (e.g., by expressing proapoptotic proteins).

The HSV amplicon particles described herein can be administered to subjects directly or indirectly, alone or in combination with other therapeutic agents, and by any route of administration. For example, the HSV amplicon particles can be administered to a subject indirectly by administering cells transduced with the vector to the subject systemically. Alternatively, or in addition, an HSV amplicon particle could be administered directly to a local target site. For example, an HSV amplicon particle that expresses a tumor-specific antigen can be introduced into a tumor by, for example, injecting the vector into the tumor or into the vicinity of the tumor (or, in the event the cancer is a blood-borne tumor, into the bloodstream).

The herpesvirus amplicon particles described herein, and cells that contain them, can be administered, directly or indirectly, with other species of HSV-transduced cells (e.g., HSV-immunomodulatory transduced cells) or in combination with other therapies, such as chemotherapy. Such administrations may be concurrent or they may be done sequentially. Thus, in one embodiment, HSV amplicon particles, the vectors with which they are made (i.e., packaging vectors, amplicon plasmids, and vectors that express an accessory protein) can be injected into a subject (e.g., a human patient) to treat, for example, cancer or an infectious disease. Thus, provided herein are compositions comprising the HSV amplicon particles and a second agent such as a chemotherapeutic, antibacterial agent, antiviral agent or the like.

As used herein, the term isolated requires that the material be removed from its original environment (e.g., the natural environment if it is naturally occurring).

As used throughout, by a subject is meant an individual. Thus, the subject can include domesticated animals, such as cats, dogs, etc., livestock (e.g., cattle, horses, pigs, sheep, goats, etc.), laboratory animals (e.g., mouse, rabbit, rat, guinea pig, etc.) and birds. For example, the subject is a mammal such as a primate, and, including, a human.

Ranges may be expressed herein as from about one particular value and/or to about another particular value. Similarly, when values are expressed as approximations, by use of the term about, it will be understood that the particular value is included. It will be further understood that the endpoints of each of the ranges are significant both in relation to the other endpoint, and independently of the other endpoint.

As used herein the terms treatment, treat or treating refers to a method of reducing the effects of a disease or condition or at least one symptom of the disease or condition. Thus, the disclosed method treatment can refer to a 10%, 20%, 30%, 40%, 50%, 60%, 70%, 80%, 90% or 100% reduction in the severity of an established disease or condition or symptom of the disease or condition. For example, the method for treating cancer is considered to be a treatment if there is a 10% reduction in one or more symptoms or clinical signs of the disease in a subject as compared to control. Thus the reduction can be a 10, 20, 30, 40, 50, 60, 70, 80, 90, 100% or any percent reduction in between 10 and 100 as compared to native or control levels. It is understood that treatment does not necessarily refer to a cure or complete ablation of the disease, condition or symptoms of the disease or condition.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that, while specific reference to each various individual and collective combinations and permutation of these compounds may not be explicitly disclosed, each is specifically contemplated and described herein. For example, if an inhibitor is disclosed and discussed and a number of modifications that can be made to a number of molecules of the inhibitor are discussed, each and every combination and permutation of inhibitor and the modifications that are possible are specifically contemplated unless specifically indicated to the contrary. This concept applies to all aspects of this disclosure including, but not limited to, steps in methods of making and using the disclosed compositions. Thus, if there are a variety of additional steps that can be performed it is understood that each of these additional steps can be performed with any specific steps or combination of steps of the disclosed methods, and that each such combination is specifically contemplated and should be considered disclosed.

Publications cited herein and the material for which they are cited are hereby specifically incorporated by reference. No admission is made that any reference constitutes prior art. The discussion of references states what their authors assert, and applicants reserve the right to challenge the accuracy and pertinency of the cited documents.

The terms control levels or control cells are defined as the standard by which a change is measured, for example, the controls are not subjected to the experiment, but are instead subjected to a defined set of parameters, or the controls are based on pre- or post-treatment levels.

### EXAMPLES

### Example 1

### Infectivity of Herpes Simplex Virus Type-1 (Hsv-1) Amplicon Vectors is Determined by the Helper Virus Strain Used for Packaging

Materials and Methods.

Expansion of clinical HSV-1 isolates. Nineteen clinical isolates of HSV-1 were obtained from the UR Clinical Microbiology Laboratory. Samples were selected from individuals with mild disease symptoms (i.e., with no evidence of encephalitis) and were provided without patient identifying information in an approximate volume of 1 ml each. One hundred microliters of each isolate were used to infect 1.1×10⁶ VERO cells in 60-mm culture dishes and incubated at 34° C. Viral propagation was assessed by monitoring apparent cytopathic effect (CPE). The length of time required by the isolates to reach 100% CPE ranged between 3 and 5 days. Each dish was then incubated at 37° C. for 2 hours to enhance viral release from the host cells. The cells and supernatants were collected and frozen at −80° C. (represented the P0 stock). Each isolate was then further expanded (P1 stock generation). The P0 stocks were thawed at 37° C., subjected to sonication to liberate any virus residing within host cells and centrifuged to remove the majority of cellular debris. The supernatants (approximately 4 ml each) were used to infect 4×10⁶ Vero cells per T-75 flask and propagation was monitored. 100% CPE was observed between 4 and 5 days of incubation. The P1 viral stocks were generated, stored in 1-ml aliquots, and frozen at −80° C. until titered by a previously described plaque-based method (Geschwind et al., Brain Res 24:327-35, 1994).

Analysis of virus growth kinetics. VERO cells were used to determine the growth kinetics of selected primary HSV-1 isolates, as well as the reconstituted F5 virus stock (Cunningham and Davison, Virology 197:116-24, 1993) and the laboratory adapted isolates, KOS (available from the American Type Culture Collection, ATCC catalog number VR-1493) and strain 17 (a non-syncytium forming, syn+ (Ruyechan et al., J Virol 29:677-97, 1979). Cells were plated at a density of 2×10⁵ cells/well in 24-well tissue culture plates. Eight time points were selected for determination of viral propagation (0, 2, 6, 12, 18, 24, 36, 48 hours) and each well was infected with 4×10⁴ pfu (MOI=0.2). Viral medium was aspirated at the conclusion of each time point and cells were lysed in 100 μl of 100 mM potassium phosphate, pH 7.8 and 0.2% Triton X-100 containing 1 mM DTT for 10 minutes at 25° C. The resulting lysates were collected and frozen at −80° C. Total DNA was obtained as described (Bowers et al., Mol Ther 1:294-9, 2000), and the resulting DNA concentration was determined by spectrophotometric analysis. Transduction analysis was performed using quantitative real-time DNA PCR (qRT-PCR) specific for the ICP0 gene of HSV-1.

Quantitative real-time PCR. Total DNA from cells infected with HSV-1 isolates was analyzed using qRT-PCR. Briefly, 12.5 ng of DNA was loaded into 25 μl PCR reactions and analyzed using the 7300 Real Time PCR System (Applied Biosystems, Foster City, Calif.). ICP0 gene copy number was determined using the pCI110 plasmid as a standard curve. Primers (Fwd: 5′-ATGTTTCCCGTCTGGTCCAC-3′ (SEQ ID NO:1)) (Rev: 5′-CCCTGTCGCCTTAC GTGAA-3′ (SEQ ID NO:2)) and probe (5′-CCCCGTCTCCATGTCCAGGATGG-3′ (SEQ ID NO:3)) were designed using the Primer Express 3.0 software (Applied Biosystems, Foster City, Calif.). Data were normalized using cellular genomic DNA (for the 18S rRNA gene) using primers (Fwd: 5′-CGGCTACCACATCCAAGGAA-3′ (SEQ ID NO:4)) (Rev: 5′-GCTGGAATTACCGCGGCT-3′ (SEQ ID NO:5)) (Probe: 5′-TGCTGGCACCAGACTTGCCCTC-3′ (SEQ ID NO:6)).

Packaging and propagation of an amplicon vector by HSV-1 isolates. Twenty T-150 flasks containing 8×10⁶ Vero cells per flask were transfected with 56 μg of pHSVlac plasmid DNA (Geller and Breakefield, Science 241:1667-9, 1988), using the Lipofectamine 2000 transfection reagent (Invitrogen, Carlsbad, Calif.). Each flask was incubated overnight at 37° C. The following day, the expanded HSV-1 clinical isolates and the reconstituted F5 clone were used to infect each flask at an MOI of 0.2. Four days later, each flask was incubated at 37° C. for 2 hours to enhance viral release from the host cells. The cells and supernatants were collected and frozen at −80° C. to create a P0 stock and further expanded to generate a P1 stock. Each P1 stock was subsequently subjected to sucrose-gradient concentration. The concentrated viral stocks were resuspended in 500 μl of DPBS containing calcium and magnesium and frozen as 50 μl aliquots at −80° C. Wild-type HSV-1 titers were determined by plaque assay on VERO cells and amplicon titers were determined using X-gal histochemistry on NIH3T3 cells as described (Bowers et al., Mol Ther 1:294-9, 2000).

Human dendritic cells. Human dendritic cells (DC) were differentiated from CD14+ monocytes, as outlined in previous studies (Maguire et al., Vaccine 24:671-82, 2006). Briefly, leukocyte concentrates received from the New York Blood Center (New York, N.Y.) or whole blood samples were layered on a LYMPHOPREP™ (Axis-Shield, Oslo, Norway) cushion and human peripheral blood mononuclear cells (PBMCs) were isolated by density centrifugation. CD14+ monocytes from buffy coats obtained from the Lymphoprep interface were enriched by positive selection with anti-CD14 MACS beads (Miltenyi Biotec, Auburn, Calif.). Monocytes were cultured in Stemline Dendritic Cell Maturation Media (Sigma, St. Louis, Mo.) supplemented with 2 mM L-glutamine, and 50 ng/ml recombinant human GMCSF (R&D Systems, Minneapolis, Minn.) and 25 ng/ml recombinant human IL-4 (R&D Systems). Media was replenished every 2 days. After 9 days in culture, the monocyte-derived human DC were used in HSV-1 amplicon transduction assays.

Amplicon transduction assays. Assessment of amplicon transduction efficiency was performed in differentiated human DC. Cells were incubated at 37° C./5% CO2 under humidified conditions. For infections, 3×10⁵ cells were seeded into 24-well plates and allowed to adhere overnight at 37° C.15% CO2. Cells were then transduced at various multiplicities of infection (MOI) ranging from 0.001 to 0.1 (unless otherwise specified) with amplicons generated using the different isolates as helper virus. At 24 hours post transduction, cultures were harvested for analysis. Transduction efficiency was assessed either by enzymatic assay for β-galactosidase activity, or by a histochemical staining method. The enzymatic assay was performed using the GalactoLite Plus kit (Applied Biosystems, Foster City, Calif.) according to manufacturer's directions. Briefly, cells were lysed with 200 μl lysis buffer supplemented with 1 mM dithiothreitol (DTT). Lysates were clarified by centrifugation at 13,000 rpm for 7 min at 4° C. and protein concentration was measured using Bradford reagent (BioRad, Hercules, Calif.). Five microliters of cleared lysate (1 μg total protein) were assayed for β-galactosidase activity. Light emission was measured in a white 96-well plate, using a luminometer (SpectraCount Version 3.0, Packard BioScience, Meriden, Conn.) and measurements of β-galactosidase activity were normalized to total protein content.

X gal histochemistry was performed by staining with X-gal substrate. Briefly, transduced cells were pelleted by low speed centrifugation, washed with PBS, and fixed for 5 min at 25° C. in a 2% formaldehyde/0.2% glutaraldehyde solution. The cells were then washed with PBS and stained with X-gal (20 mg/ml X-gal; Sigma) in dimethyl sulfoxide (DMSO), in KFe(CN)/PBS solution. Cells were then incubated at 37° C. for 45 min, and observed using phase-contrast light microscopy using an Olympus IX81 inverted fluorescent microscope. Images were acquired using a CCD digital photo camera, and then evaluated using Image Pro Plus software (version 4.5.1). Statistical analyses were performed using GraphPad Prism software.

Results

Primary HSV-1 isolates vary in their ability to propagate amplicon vectors. Described below is the comparison of the biological properties of HSV-1 amplicon stocks generated using a panel of primary HSV-1 isolates with those of an amplicon stock generated using a reconstituted, molecularly cloned virus stock that is widely used in the production of helper-free amplicon particles (designated here as F5) (Cunningham and Davison, Virology 197:116-24, 1993; Stavropoulos and Strathdee, J Virol 72:7137-43 1998). This example shows that minimally passaged clinical HSV-1 isolates permit the generation of amplicon stocks with more desirable properties (e.g., expanded host range) than is possible using the current helper virus genome.

Virus stocks were generated that contained a co-propagated amplicon vector encoding a β-galactosidase transcription unit (so as to allow convenient assessment of virally-mediated gene transfer into cultured target cells of interest). The ability to efficiently package and propagate amplicon stocks is an important criterion with respect to identifying new HSV-1 strains for use as helper viruses in the generation of amplicon stocks. The ability of a panel of HSV-1 primary isolates to propagate amplicon stocks was determined. To do this, amplicon-containing stocks were generated using each of the various isolates. Functional assays were then used to separately measure the titer of the helper virus and the amplicon vector. Helper virus was quantitated by measuring virus plaque forming units (PFU) in VERO cells, while amplicon was titered by measuring β-galactosidase expressing, blue-forming units (BFU) in 3T3 cells. The ratio of amplicon:helper virus was then determined, and the results are presented in Table 1. This analysis revealed that the primary HSV-1 isolates varied in their ability to propagate amplicon stocks, but that several of them outperformed the F5 virus stock in this regard.

For Table 1, amplicon-containing stocks were generated using each of the various primary HSV-1 isolates. Wild-type (helper virus) titers were determined by plaque assay on VERO cells (pfu/ml) and amplicon titers were determined using X-gal histochemistry on NIH 3T3 cells (blue forming units (bfu)/ml). The ratio of amplicon:helper virus was then determined. The results show that most of the primary isolates were able to efficiently propagate the amplicon plasmid, expect for isolates 1 and 10.

TABLE 1
Ability of primary HSV-1 isolates to package
and propagate amplicon stocks.

| | Amplicon:Helper | Amplicon Titer | Helper Titer |
|---|---|---|---|
| Isolate | Ratio | (×10⁷ bfu/ml) | (×10⁷ pfu/ml) |
| 1 | 0.086 | 3 | 34.8 |
| 2 | 0.363 | 27 | 74.4 |
| 3 | 0.704 | 79.5 | 113 |
| 4 | 0.560 | 48 | 85.8 |
| 5 | 0.680 | 51 | 75 |
| 6 | 0.233 | 24 | 103 |
| 7 | 0.319 | 34.5 | 108 |
| 8 | 0.447 | 25.5 | 57 |
| 9 | 0.464 | 42 | 90.6 |
| 10 | 0.074 | 8.7 | 114 |
| 11 | 0.450 | 30 | 66.6 |
| 12 | 0.526 | 30 | 57 |
| 13 | 0.443 | 12.5 | 28.2 |
| 14 | 0.495 | 33 | 66.6 |
| 15 | 0.417 | 6.75 | 16.25 |
| 16 | 0.482 | 30 | 63.6 |
| 17 | 0.386 | 28.5 | 73.8 |
| 18 | 0.490 | 7.05 | 14.4 |
| 19 | 1.01 | 52.5 | 52.2 |
| F5 | 0.470 | 28.5 | 60.6 |

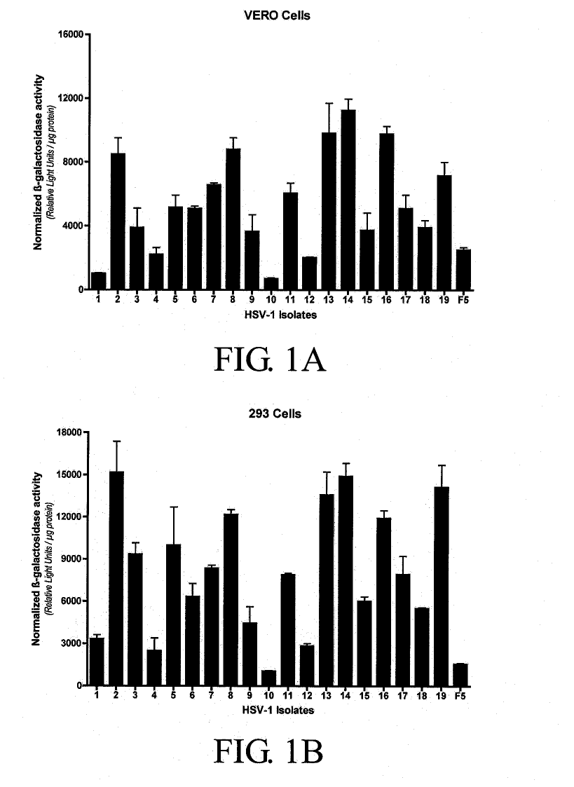

Primary HSV-1 isolates vary in their ability to infect established cell lines. To examine the biological properties of amplicon stocks packaged by the panel of clinical isolates, or the F5 control strain, two established cell lines (VERO and 293 cells) were exposed to helper-containing amplicon stocks at a MOI of 0.1 (in this experiment, and subsequent experiments, the infecting MOI was defined in terms of the titer of the lacZ-encoding amplicon vector, as measured in VERO cells; see Methods above). Cultures were then harvested at 24 hours post infection and β-galactosidase activity was assayed from cell lysates. The results showed that amplicon stocks packaged by most of the clinical isolates were able to elicit higher levels of gene expression in both VERO (FIG. 1A) and 293 cells (FIG. 1B), when compared to the F5 virus stock.

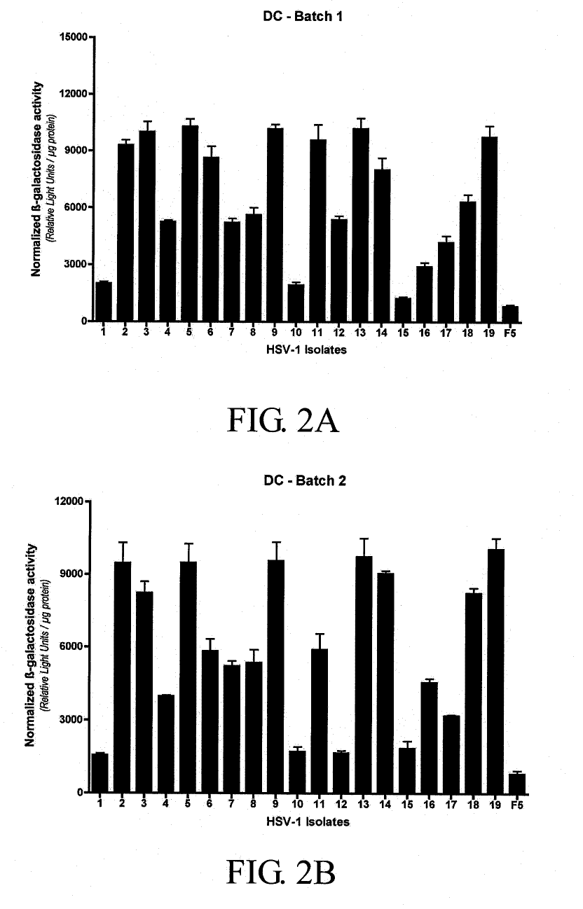

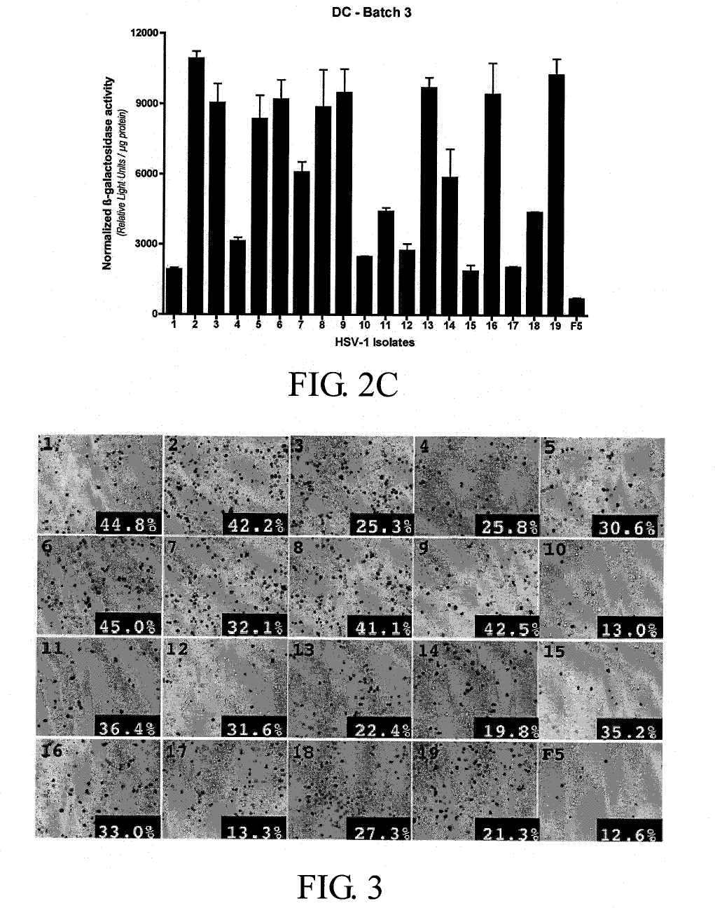

Primary HSV-1 isolates vary in their ability to infect monocyte-derived human DC. By using different HSV-1 isolate strains to package amplicon particles, provided herein are amplicon stocks able to transduce biologically important cell types like dendritic cells. Human monocyte-derived cultured DC were exposed to amplicon-containing virus stocks derived from each of the 19 primary isolates and from the F5 strain, at an MOI of 0.1. Twenty-four hours later, cells were harvested and analyzed. Quantitation of β-galactosidase activity in cell lysates (FIGS. 2A, 2B and 2C), showed that amplicon vectors packaged by the primary HSV-1 isolates varied in their ability to transduce DC, but that the great majority of the primary isolates were able to significantly outperform the F5 virus stock, in terms of their ability to generate amplicon particles that could efficiently transduce DC. To confirm that differences in DC transduction efficiency were reproducible, and not a reflection of a specific donor, this analysis was repeated using DC that were isolated from multiple donors. This analysis revealed very similar findings, irrespective of the source of the DC (FIG. 2).

Finally, β-galactosidase expression was assayed using a histochemical staining method (FIG. 3). This allowed visualization of individual β-galactosidase positive cells. As a result, the data show that increased levels of β-galactosidase activity measured in the GalactoLight assay (FIG. 2) were also associated with an increase in the number of β-galactosidase-positive cells. Therefore, increased levels of β-galactosidase expression measured in the GalactoLight assay can be attributed, to an increase in the percentage of the dendritic cell population that became transduced by the amplicon vector (and not simply because of an increase in the per-cell level of reporter gene expression). Consistent with this, there was a statistically significant correlation between the level of β-galactosidase expression, as measured by the GalactoLight assay versus the histochemical staining method; Pearson r=0.506, p<0.05.

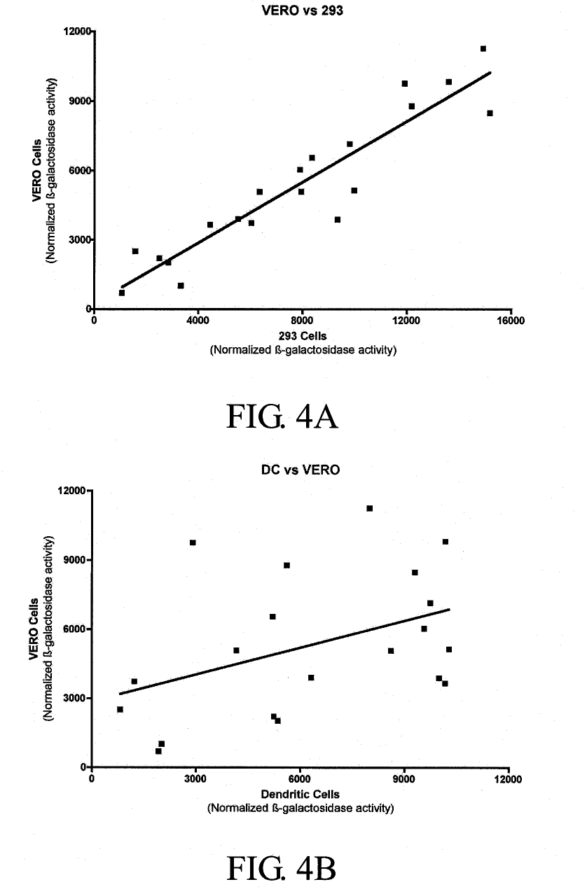

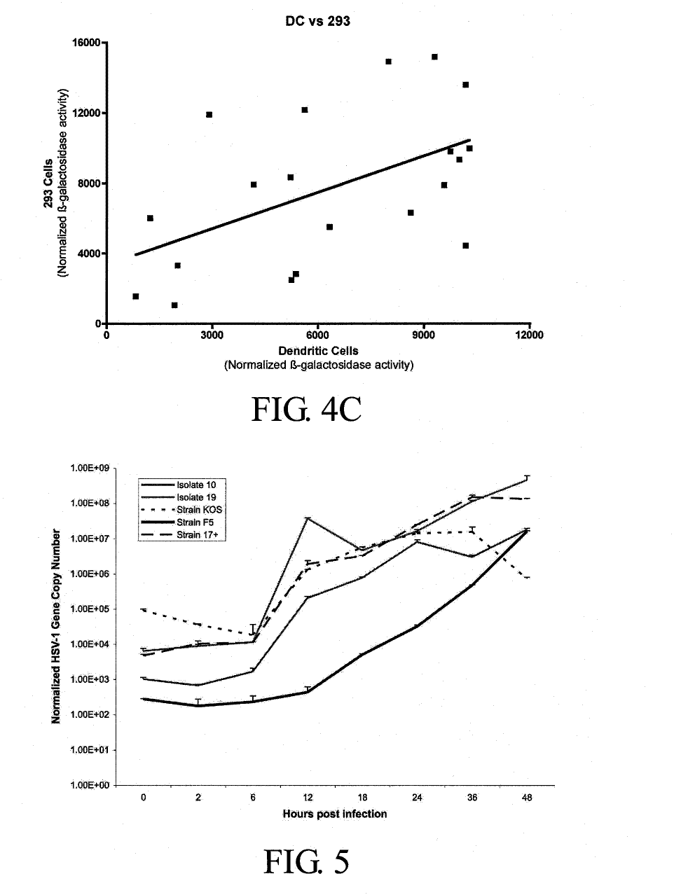

Amplicon-mediated gene expression in VERO cells correlates strongly with expression in 293 cells but more weakly with expression in DC. One possible outcome of using different HSV-1 strains to package amplicon stocks is that there may be variation in the ability of the resulting amplicon particles to transduce different cell types. Therefore linear regression analysis was conducted of cell transduction data for the VERO and 293 cell lines, and the primary dendritic cells. The associations between gene expression levels (averaged over three replicates) were examined in a pairwise fashion for the three different cell types using linear regression and correlation analysis. FIG. 4 shows the graphical results of this analysis, while Table 2 provides a statistical summary of the results. As noted in Table 2, there was a very strong, highly significant correlation between the magnitude of amplicon-mediated gene expression in the two cultured cell lines (VERO cells and 293 cells); Pearson r=0.934, 95% confidence interval 0.838 to 0.974, p<0.0001. In contrast, the association between lacZ gene expression levels in VERO cells and primary DC was somewhat weaker and failed to achieve statistical significance (Pearson r=0.423, 95% confidence interval −0.024 to 0.729, p=0.063). Similarly, the association between lacZ gene expression levels in 293 cells and primary DC was also relatively modest, although statistically significance (Pearson r=0.525, 95% confidence interval 0.107 to 0.785, p=0.018).

For Table 2, a pairwise correlation analysis of cell transduction data is presented, for the VERO and 293 cell lines, and the primary dendritic cells. The associations between gene expression levels were examined in a pairwise fashion for the three different cell types (as noted in the column headings) using Pearson correlation coefficients (r). The data that were used in these analyses correspond to the datasets shown in FIG. 2 and FIG. 3 (DC Batch 1). The results show a very strong correlation between amplicon transduction efficiency in the two cultured cell lines (VERO, 293), but weaker correlations between cell line transduction efficiency and the efficiency of amplicon-mediated gene expression in primary dendritic cells (DC).

TABLE 2
Pair-wise correlation analysis of cell transduction data

| | 293 v VERO | 293 v DC | DC v VERO |
|---|---|---|---|
| Correlation | 0.934 | 0.525 | 0.4232 |
| coefficient | | | |
| (Pearson r) | | | |
| 95% confidence | 0.838 to 0.974 | 0.107 to 0.785 | −0.024 to 0.729 |
| interval | | | |
| (for Pearson r) | | | |
| P value | <0.0001 | 0.018 | 0.063 |

Formal tests were performed for equality of the correlation coefficients for the VERO/293 cell comparison, and the VERO/DC and 293/DC comparisons. Because the correlations are statistically dependent, T2 statistic originally due to Williams (Williams, J Roy Statist Soc Series B 21:396-9, 1959) and described by Steiger (Steiger, Psychol. Bull. 87, 245-251, 1980) was used for these comparisons. The results revealed that the correlation between the magnitude of amplicon-mediated gene expression in the two cultured cell lines (VERO cells and 293 cells, r=0.934) was significantly different from the other two correlations (VERO cells and DC, 293 cells and DC, p<0.0001 in each case).

Comparison of growth kinetics and biological properties of primary HSV-1 isolates versus laboratory-passaged viruses. The growth kinetics and biological properties of a representative subset of the primary HSV-1 isolate panel were compared directly to those of both a reconstituted, molecularly cloned virus stock (designated here as F5) and also to laboratory-passaged HSV-1 isolates, including both HSV-1 KOS and strain 17 (the isolate that was molecularly cloned in E. coli, and then used to produce the F5 virus stock).

The replication kinetics of two representative clinical HSV-1 isolates (1, 10) as well as the F5 stock and the laboratory isolates HSV-1 KOS and strain 17 were characterized in VERO cells. Cells were infected with virus stocks at a MOI of 0.2 (defined in terms of the infectious virus titer in VERO cells). Cultures were then harvested at predetermined time points and total DNA from infected cells was collected, and analyzed using a quantitative DNA PCR assay to measure ICP0 gene copy number. As shown in FIG. 5, there were no significant differences in the replication kinetics of the primary and laboratory-adapted virus isolates. However, the molecularly cloned F5 strain replicated with delayed kinetics and to relatively low titers when compared to the other strains (FIG. 5).

FIG. 5 also shows that there was variation in the amount of viral DNA bound to the host cells at time zero (immediately after addition of virus and washing of the cells). Since a fixed number of infectious particles (PFU) was added to the VERO cells, this difference can be attributed to a difference in the genome (particle) to infectivity ratio for the various strains (KOS >8,10,19, 17+>F5).

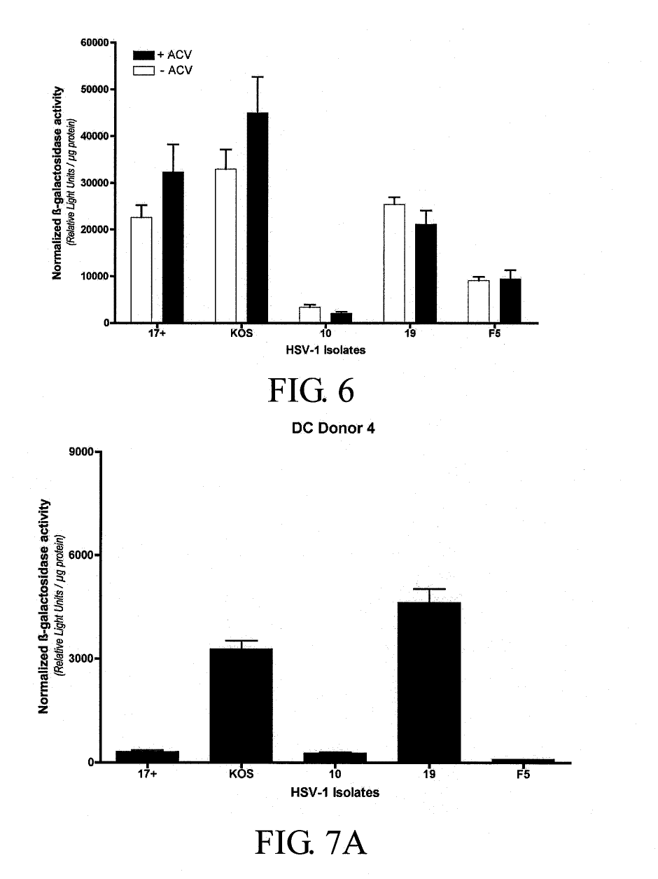

The biological properties of amplicon stocks packaged by this same panel of primary and laboratory isolates in 293 cells were compared. To do this, cells were exposed to helper-containing amplicon stocks at a MOI of 0.1. Cultures were then harvested at 24 hours post infection and β-galactosidase activity was assayed from cell lysates. The results showed that amplicon stocks packaged by primary isolate 19 efficiently transduced 293 cells, while stocks packaged by primary isolate 10 or the F5 strain were inefficient at transducing 293 cells (FIG. 6; these results are consistent with data shown in FIG. 2). Amplicon stocks packaged by the two laboratory-passaged isolates (KOS, strain 17) were also efficient at transducing 293 cells (FIG. 6). This is consistent with the adaptation of these isolates to growth in continuous cell lines.

In order to confirm that observed differences in the levels of β-galactosidase expression at the 24 hour time point were not affected by differences in helper virus replication kinetics (and accompanying replication of amplicon genomes), an additional control was included in this analysis. Specifically, the experiment was performed in the presence and absence of acyclovir (ACV), at a dose of 1 μg/ml (approx. 4.4 μM); this exceeds the IC99 for most primary HSV-1 isolates (Elion et al., PNAS 74:5716-20, 1977).

As shown in FIG. 6, the levels of β-galactosidase expression were similar, either in the presence or absence of ACV. Therefore, at the early time point used in the experiments (24 hours), β-galactosidase is being produced exclusively off transcripts that derive from the original incoming amplicon genomes and newly synthesized amplicon genome template makes no significant contribution to β-galactosidase protein production at this time point.

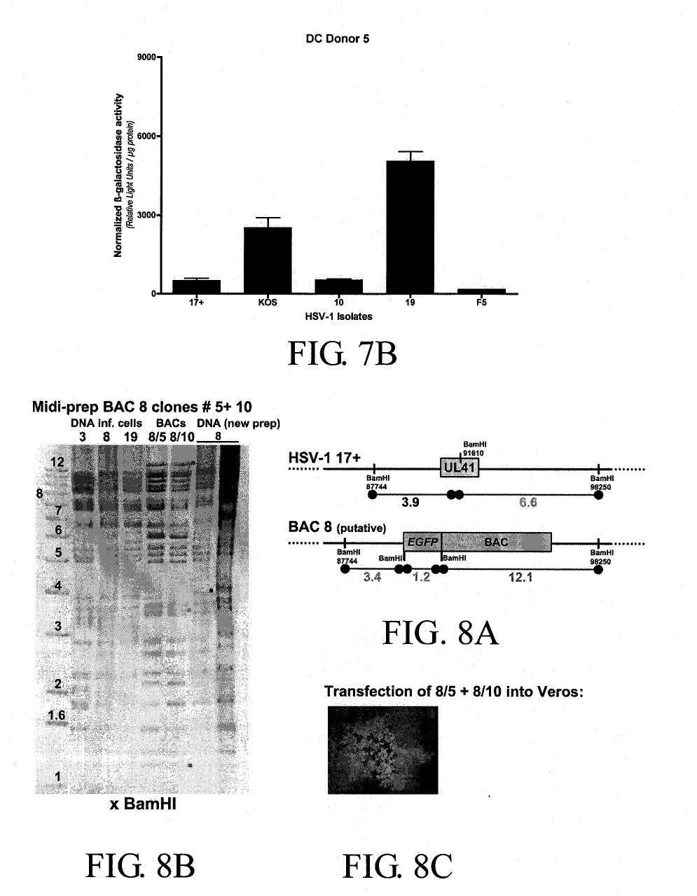

Finally, the ability of amplicon stocks packaged by primary and laboratory isolates of HSV-1 to transduce primary dendritic cells were compared. The results showed that amplicon stocks packaged by primary isolate 19 were the most efficient at transducing DC, followed by stocks packaged by the lab-adapted isolate KOS (for both donors, there was a statistically significant difference in results for primary isolate 19 versus the KOS strain; FIG. 7). Other amplicon stocks, including those packaged by primary isolate 10 as well as the molecularly cloned F5 virus and the parental lab-adapted strain 17 were uniformly inefficient at transducing DC (FIG. 7).

Molecular cloning of primary HSV-1 isolates that efficiently transduce DC. HSV-1 isolates 3, 8 and 19 all of which efficiently transduce cultured DC were molecularly cloned. To do this, a GFP marker gene and bacmid cassette were inserted into a non-essential viral gene by homologous recombination. Full-length virus genomes were then recovered into E. coli host cells and screened by restriction digestion (FIG. 8B). Finally, the infectivity of the final clones was confirmed by transfection of BAC DNA into VERO cells, followed by plaque assay. Results for isolate 8 are shown (FIG. 8C). Similar data were obtained for isolates 3 and 19.

The packaging sequences (α-sequences) from each of three HSV BACs (one each for clones 3, 8 and 19) were deleted. This eliminated the ability of the molecular clones to give rise to infectious virus progeny. Each of these BACs was able to efficiently package a reporter gene-encoding amplicon plasmid giving rise to helper-free amplicon stocks with titers equivalent to those obtained using the V2 bacmid that is employed in standard amplicon packaging protocols. The V2 bacmid was derived by reassembly of the F5 cosmid panel into a single BAC, followed by removal of the virus packaging sequences. Table 3 shows titers for amplicon stocks produced using these new, packaging-defective HSV-1 molecular clones.

TABLE 3
Titers for Amplicon Stocks Produced by HSV-1 Molecular Clones.

| | Amplicon titer |
|---|---|
| | (HSV:lacZ) (titer determined in 3T3 |
| | cells and reported in |
| HSV-1 packaging construct | expression units; lacZ+ cells) |
| V2 (standard; a-deleted, | 5.9 × 10⁷ EU/ml |
| strain 17-derived) | |
| BAG 3 (a-deleted; derived | 6.4 × 10⁷ EU/ml |
| from primary isolate 3) | |
| BAC 8 (a-deleted; derived | 1.1 × 10⁸ EU/ml |
| from primary isolate 8) | |
| BAC 19 (a-deleted; derived | 3.9 × 10⁷ EU/ml |
| from primary isolate 19) | |

### Example 2

### Amplicon Mediated Gene Transfer in CLL Cells

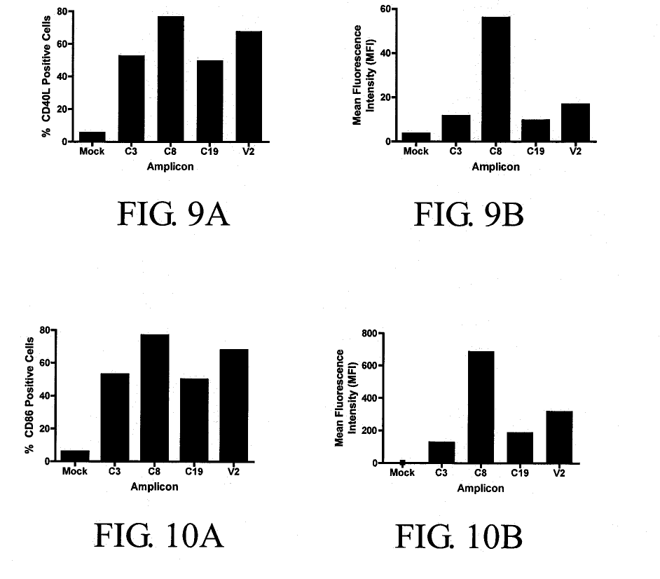

FIGS. 9A, 9B, 10A and 10B show transduction of chronic lymphocytic leukemia (CLL) cells by HSV amplicon vectors packaged using HSV-1 helper bacmids. Amplicon vectors encoding mCD40L (FIGS. 9A and 9B) or CD86 (FIGS. 10A and 10B) were packaging using HSV-1 helper bacmids C3, C8, C19 or V2 (referred to in Table 3 as BAC 3, BAC 8, BAC 19 and V2, respectively). The resulting helper-free vector stocks were used to transduce CLL cells at a multiplicity of infection (MOI) of 0.3. Twenty hours later, cells were stained with antibodies directed against mCD40L or CD86, and amplicon-mediated gene expression was the measured by flow cytometric analysis. Results are presented as the percentage of antigen positive cells (FIGS. 9A and 10A), and also as the mean fluorescence intensity (MFI) of antigen staining (FIGS. 9B and 10B). The data show that amplicon particles packaged using the C8 bacmid were considerably more efficient at transducing CLL cells than amplicon particles packaged using the other HSV-1 helper bacmids.

A number of aspects of the amplicon particles and related compositions and methods have been described. Nevertheless, it will be understood that various modifications may be made. Accordingly, other aspects are within the scope of the following claims.

1. An HSV amplicon particle comprising an amplicon vector and packaging components, wherein the packaging components are derived from a primary HSV isolate and wherein the HSV amplicon particle is helper-free.

2. The HSV amplicon particle of claim 1, wherein the primary HSV isolate is capable of producing amplicon particles that transduce dendritic cells.

3. The HSV amplicon particle of claim 1, wherein the packaging components include an envelope, a tegument and a capsid.

4. The HSV amplicon particle of claim 1, wherein the amplicon vector further comprises an expressible transgene.

5. The HSV amplicon particle of claim 4, wherein the transgene encodes a therapeutic product.

6. The HSV amplicon particle of claim 5, wherein the therapeutic product is a protein or RNA molecule.

7. The HSV amplicon particle of claim 6, wherein the RNA molecule is selected from the group consisting of antisense RNA, RNAi, and an RNA ribozyme.

8. The HSV amplicon particle of claim 5, wherein the therapeutic product is an antigen.

9. The HSV amplicon particle of claim 8, wherein the antigen is selected from the group consisting of a tumor-specific antigen, an antigen of an infectious agent and an antigen of a protein aggregate.

10. The HSV amplicon particle of claim 9, wherein the tumor-specific antigen is a prostate cancer tumor-specific antigen.

11. The HSV amplicon particle of claim 9, wherein the infectious agent is HIV.

12. The HSV amplicon particle of claim 9, wherein the protein aggregate is a protein aggregate associated with Alzheimer's disease.

13. A method for producing HSV amplicon particles, comprising co-transfecting a host cell with an amplicon vector comprising an HSV origin of replication and an HSV cleavage/packaging signal and at least one packaging vector, wherein the packaging vector is derived from a primary HSV isolate, wherein the co-transfection step is performed under conditions that result in production of the HSV amplicon particles in the host cell.

14. The method of claim 13, further comprising isolating the HSV amplicon particle from the host cell.

15. The method of claim 13, wherein the amplicon vector further comprises an expressible transgene.

16. The method of claim 15, wherein the transgene encodes a therapeutic product.

17. The method of claim 16, wherein the therapeutic product is a protein or RNA molecule.

18. The method of claim 17, wherein the RNA molecule is selected from the group consisting of antisense RNA, RNAi, and an RNA ribozyme.

19. The method of claim 16, wherein the therapeutic product is an antigen.

20. The method of claim 19, wherein the antigen is selected from the group consisting of a tumor-specific antigen, an antigen of an infectious agent and an antigen of a protein aggregate.

21. The method of claim 14, wherein the packaging vector lacks an HSV oriL origin of replication.

22. The method of claim 14, wherein the packaging vector lack an HSV cleavage/packaging signal.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. A method of treating cancer in a subject comprising administering to the subject the amplicon particles of claim 4, wherein the transgene encodes a tumor-specific antigen.

36. The method of claim 35, wherein the cancer is prostate cancer.

37. A method of treating a disease caused by an infectious agent in a subject comprising administering to the subject the amplicon particles of claim 4, wherein the transgene encodes an antigen of the infectious agent.

38. The method of claim 37, wherein the infectious agent is HIV.

39. A method of treating a protein aggregate disorder comprising administering to the subject the amplicon particles of claim 4, wherein the transgene encodes an antigen of the protein aggregate.

40. The method of claim 39, wherein the protein aggregate disorder is Alzheimer's disease.

41. A method for selecting a primary HSV isolate for use in a method of producing HSV amplicon particles comprising:
a) co-transfecting a host cell with an amplicon vector comprising an HSV origin of replication and an HSV cleavage/packaging signal and a candidate primary HSV isolate to be tested, under conditions that allow for production of at least one HSV amplicon particle in the host cell;
b) isolating the amplicon particle from the host cell;
c) contacting the amplicon particle with at least one dendritic cell; and
d) determining whether the amplicon particle transduces the dendritic cell, wherein transduction of the dendritic cell by the amplicon particle indicates that the primary HSV isolate is suitable for use in the method of producing HSV amplicon particles.
